(19) United States
(12) Plant Patent Application Publication
(10) Pub. No.: US 2003/0014791 P1
(43) Pub. Date: Jan. 16, 2003

(54) Grapevine '12-76-71'

(76) Inventor: Harold P. Olmo, Davis, CA (US)

Correspondence Address: Harold P. Olmo, 6964 Vineyard Lane, Davis, CA, 95616, US

(21) Appl. No.: 09/891,425

(22) Filed: Jun. 27, 2001

(51) Int. Cl. A01H 5/00

(52) U.S. Cl. PLT/205

(57) ABSTRACT

This new variety ‘12-76-71’, a red, seedless, table grape, is unique because of its combination of strong muscat flavor, seedlessness and high productivity. It closely resembles ‘Flame Seedless’ table grape which is one of its progenitors, but is different because of its later ripening and muscat flavor. The stenospermic fruit has muscat flavor, very firm flesh and is very sweet at maturity. Additionally, the fruit retains its form and quality over a long period of preservation in handling, storage and marketing.

### PLANT CHARACTERISTICS

The new muscat table grapevine ‘12-76-71’ of vinifera parentage resulted from the cross ‘Redglobe’×an unnamed red, seedless grape ‘7-7-2’ made at McFarland, Califa. in 1992. The new grapevine has been multiplied by cuttings as well as propagated by grafting on several rootstocks. These plants proved stable and were typical of the original vine. The invention is a unique seedless table grape with muscat flavor.

In the drawings:

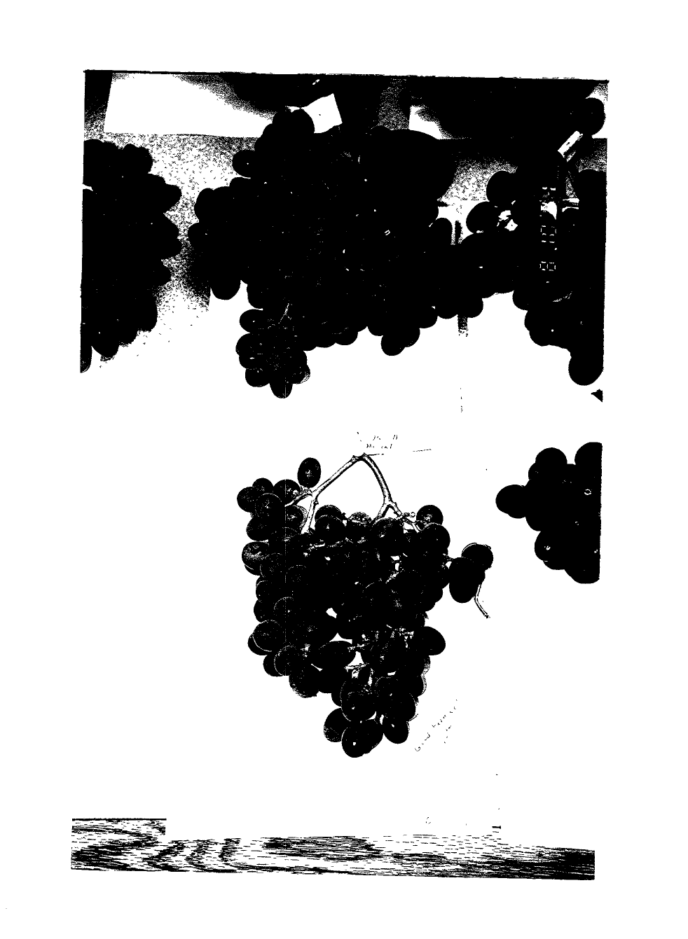

FIG. 1 Fruit clusters at harvest.

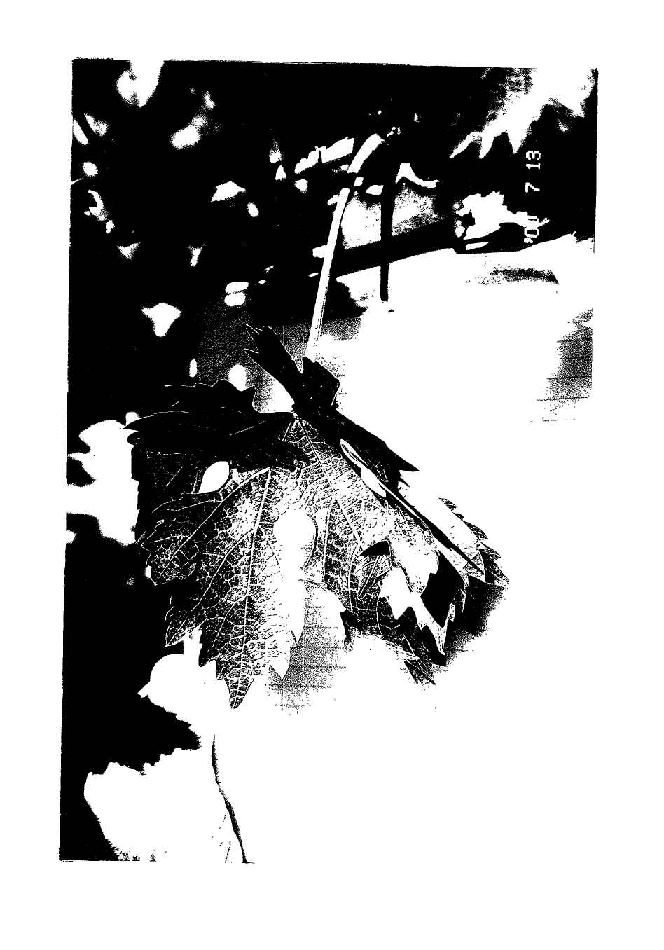

FIG. 2 Leaf, upper surface

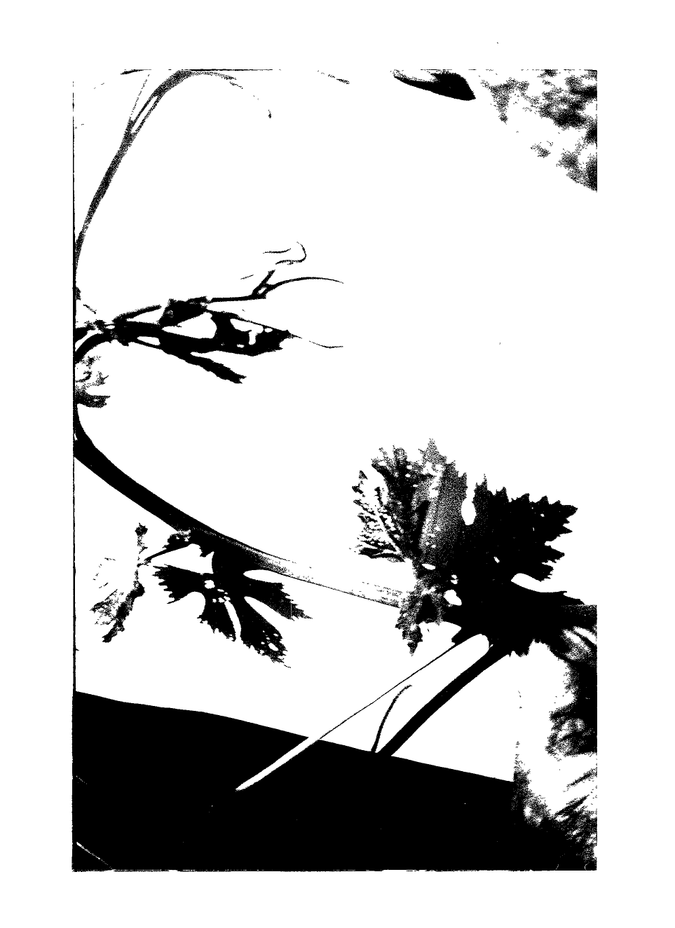

FIG. 3 Young shoot and leaves.

Vine: Vigorous, but growth compact because of short basal internodes, fruiting area crowded if short pruned. Inflorescences usual on nodes 3 and 5 and require selective removal to insure best form and size of clusters.

Shoot tip (10-12”): Shoot tip bronzed with light hairiness, first flat leaf glabrous.

Cane: Large, straight, vigorous; very long intemodes, often one cm. in diameter and ten cm. long, surface ridged with continuous dark brown striations; cross section circular; dormant buds large, flattened, tightly sealed; strong lateral branching. Basal buds very fruitful.

Leaf: Outline spherical, 5 lobed, marginal teeth large, acute. Medium in size, supper surface dark green, unique shape created by inward curvature of basal portion of ribs, causing marked infolding of lobes to almost tubular shape. When leaf is flattened, the wide lobes overlap to close the deep, narrow sinuses. Petiole slender, with marked striations of maroon color, often longer than blade, petiolar sinus wide, open U.

Cluster: Primnary clusters average 375 gm. The largest berries average about 8 gm. Total number of berries per cluster is in the range of 50-70. Harvest time at Delano, Califa, is around the 28^{th} of July when the fruit reaches 18-20° Balling, the acidity is quite low and the muscat flavor is evident and appreciated by the consumer.

Branches: Lateral branches are slender and irregular in length and position on the central axis, yet very wiry and resistant to breakage. Flowers and fruit set are mostly concentrated toward the terminal portions of the lateral branches, leaving more exposure in the basal framework and making identity of a central axis difficult. The result is a wide variation in cluster shape and size and failure to repeat a common conical outline.

Berries: Spherical, skin dark red to reddish black, forming stenospermically with little evidence of seed traces. Appearance of berries at harvest is uniform for size and shape. The dark red coloring of the skin surface is also more consistent and complete because of the spacing of the berries to allow adequate exposure to light. Although the epidermal skin is very thin, it is very resistant to cracking and abrasion. Considering the large size of the berry, it is securely attached to the capstem (pedicel) and at harvest and marketing has a low incidence of shatter.

The resistance of fruit to abrasion and water loss provides a longer period of cold storage as well as extended shelf life without refrigeration. These characteristics are best explained by the unique anatomical development of the berry in which the vascular system of the core replaces and fills the space or cavity normally occupied by the developing seeds. The peripheral vascular strands likewise proliferate and contribute to the very firm structure of the berry flesh, a characteristic not encountered in muscat varieties.

### ORIGIN AND ASEXUAL REPRODUCTION OF THE NEW VARIETY

The present variety of grapevine hereof resulted from a long term table grape breeding project of Marko Zaninovich, Inc.; Delano, Califa. In this case, emphasis centered on the development of a high quality, seedless, muscat flavored table grape. The sequence of the parentage is shown below:

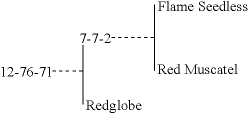

We claim:

1. The new and distinct variety of TABLE GRAPE herein described and illustrated and identified by the characteristics enumerated above.
